(19) United States
(12) Patent Application Publication
(10) Pub. No.: US 2017/0007760 A1
(43) Pub. Date: Jan. 12, 2017

(54) INJECTION DEVICE

(71) Applicant: IDTM GMBH, Castrop-Rauxel (DE)

(72) Inventors: Michael Friebe, Recklinghausen (DE); Stefan Siegfried Hans-Georg Hellwig, Hemer (DE)

(21) Appl. No.: 15/117,741

(22) PCT Filed: Jan. 21, 2015
(86) PCT No.: PCT/EP2015/051135

§ 371 Date: Aug. 10, 2016

(30) Foreign Application Priority Data
Feb. 12, 2014 (DE) 10 2014 101 763.3

(51) Int. Cl. A61M 5/00; A61M 5/145; G01R 33/56; A61B 5/055; A61B 6/03; G01R 33/28; A61M 5/20; A61M 39/22

(52) U.S. Cl. CPC A61M 5/007; A61M 5/2053; A61M 5/14526; A61M 39/22; A61B 5/055; A61B 6/032; G01R 33/285; G01R 33/5601; A61M 2039/265

(57) ABSTRACT

Injection device (1, 1′), in particular for injecting a liquid into a body in magnetic resonance tomography, comprising: a syringe with a reservoir (3) for holding a liquid, with a nozzle (5) arranged on the reservoir, and with a plunger (9) for expelling the liquid from the reservoir through the nozzle, a drive member, which is connected to the plunger (9) in order to drive the plunger (9), and a non-electrical energy accumulator (17, 17′), which is connected to the drive member in order to operate the drive member.

### FIELD OF THE INVENTION

The invention relates to an injection device for injecting a liquid during a diagnostic tomography, in particular an MRT examination or CT examination.

### PRIOR ART

Injection devices are used in magnetic resonance tomography (MRT) and computed tomography (CT) in order to inject a contrast agent and thereby obtain images of a patient that are able to be used for purposes of diagnosis. Known injection devices for MRT are generally arranged at some distance from the magnetic resonance tomograph. Typically, the injection devices are arranged with the magnetic resonance tomograph in a special room which meets special electromagnetic requirements.

It is known that, in order to administer the injection substance, a person enters the MRT room and administers the injection substance. Moreover, it is known to provide special injection devices that can be controlled remotely. To permit the remote control, the injection devices can contain large amounts of magnetic materials, which are attracted during the operation of the magnetic resonance tomograph.

Therefore, such injection devices need to have a certain minimum distance from the coils of the MRT. This is done in an attempt to ensure that the quality of the image or of the tomogram of the MRT system is not adversely affected by the operation of the injection device. Relatively long lines are therefore needed from the injector head to the patient. This is disadvantageous for injection, since large amounts of irrigation solution have to be used and long hoses are required. This also complicates handling. Moreover, if the long hoses are to be replaced for each patient, a lot of waste builds up, which has to be specially disposed of.

DE 10 2006 006 952 A1 discloses an injector system with a hydraulic pump, but this system, on account of the pump, is still relatively complex and requires long lines.

### DISCLOSURE OF THE INVENTION

The object of the invention is to make available an improved injection device which does not impair or only slightly impairs the operation of the MRT. Reliable administration of injection substance is also desirable.

The object is achieved with an injection device as claimed in claim 1 and with a magnetic resonance tomograph as claimed in the additional independent claim. Typical developments are contained in the dependent claims.

A first aspect of the invention relates to an injection device, in particular for injecting a liquid into a body in magnetic resonance tomography, comprising: a syringe with a reservoir for holding a liquid, with a nozzle arranged on the reservoir, and with a plunger for expelling the liquid from the reservoir through the nozzle, a drive member, which is connected to the plunger in order to drive the plunger, and a non-electrical energy accumulator, which is connected to the drive member in order to operate the drive member.

Typical embodiments afford the possibility of placing an injection device close to a magnet of a magnetic resonance tomograph, wherein the arrangement of a non-electrical energy accumulator reduces the possibility of attack for electromagnetic radiation. The force applied to the energy accumulator is thus dependent on the amount and nature of the magnetizable material that it contains. In embodiments of the invention, non-electrical energy accumulators are used. In this way, both the relevant mass of the energy accumulator for the magnetic forces and also the magnetically relevant mass in other parts of the injection device can be reduced, for example in a drive member which can typically be operated non-electrically, e.g. a turbine acted on by fluid, in particular gas, or a pneumatically operated pressure piston.

Typical non-electrical energy accumulators comprise a store for compressed gas or a spring. In typical illustrative embodiments with an energy accumulator designed as a store for compressed gas, the energy accumulator is connected to the drive member via a fluid line. The fluid line is typically made of a non-metallic material, for example of plastic. In further embodiments, the fluid line is composed of a metallic but preferably non-magnetic material, for example in embodiments with a comparatively short fluid line between the energy accumulator and the drive member.

Typical embodiments comprise fluid lines between the energy accumulator and the drive member with a length of less than 1 m or less than 50 cm. In typical embodiments with a store for compressed gas as energy accumulator, the drive member is designed to effect a movement of the plunger with the aid of compressed gas from the fluid line.

In typical embodiments, the store for compressed gas is connected by a screw connection to the fluid line or to the valve. Connected to the fluid line or connected to a line via a thread. In this way, the store for the compressed gas can be replaced easily and in an uncomplicated way. This also permits very small stores, since they do not have to be used for numerous applications.

Typically used compressed gases in embodiments comprise or at least substantially consist of carbon dioxide, nitrogen or helium. In the store, the gas can be compressed to pressures of at least 10 times, at least 20 times or at least 50 times the atmospheric pressure at standard conditions. In the store, gas in typical embodiments is compressed to a pressure of at least 1 MPa, at least 2 MPa or at least 5 MPa. This affords the advantage that only small cross-sectional areas are needed in the drive member for driving the plunger.

Typical embodiments comprise a valve, which is arranged between the energy accumulator and the drive member. Typical valves include valves that can be opened only once by means of the compressed gas, or valves that are able to open with the aid of the compressed gas and then close and, if appropriate, re-open. Embodiments with valves that are able to open once afford the advantage of being simple to construct. In such embodiments, after the valve has been released, all or at least most of the content of the reservoir of the syringe is forced out. Injection devices with valves that open and close again afford the advantage that injections can be divided up into multiple injections, or else partial amounts of liquids in the reservoir can be injected.

Typical valves of injection devices can be controlled electromagnetically. Electromagnetically controllable valves afford the advantage that they can be easily remotely controlled and are reliable. In further embodiments, the valves can be controlled pneumatically, for example by air pressure. Since, in order to open the valve, far less force or work generally has to be expended than has to be expended for driving the plunger, a remotely controlled valve can also be made smaller than the drive member of the plunger. In typical embodiments, the distance from the energy accumulator to the drive member is less than 1 m, typically less than 0.50 m. This affords the advantage that a compact appliance can be created which is able to be installed near the electrotomography unit or the magnet of the electrotomography unit.

Typical embodiments comprise a spring as energy accumulator. Typically, the spring is connected directly or indirectly to the plunger, a kinematic connection being present in typical illustrative embodiments. This affords the advantage of direct coupling of the spring to the plunger.

Typical embodiments comprise an actuator for releasing the spring. Upon release of the spring by means of the actuator, the spring is freed such that it drives the plunger, and liquid is thus forced out of the reservoir. Typically, the actuator is electromagnetic or can be controlled electromagnetically. Further actuators in typical illustrative embodiments are pneumatically controllable.

An aspect of the invention relates to a tomograph, in particular a magnetic resonance tomograph or computed tomograph, with an injection device as claimed in one of the preceding claims.

Typically, in embodiments according to the invention of magnetic resonance tomographs, the syringe and the energy accumulator are arranged on a housing of coils of the magnetic resonance tomograph. This affords the advantage that the lines for injecting the liquid from the syringe to the patient can be kept short.

In further embodiments, an additional frame is used for the housing of the coils of the magnetic resonance tomograph in order to receive the syringe or the energy accumulator, typically both. This affords the advantage of variable handling options. In typical illustrative embodiments, the syringe and the energy accumulator are at a maximum distance of 1 m, typically less than 0.7 m or typically less than 0.5 m from the housing of the coils of the magnetic resonance tomograph. In further embodiments, the syringe and the energy accumulator are at a maximum of 2.0 m or a maximum of 1.5 m or a maximum of 1.0 m from the isocenter of the coils of the magnetic resonance tomograph. In typical embodiments, an arrangement of the syringe and of the energy accumulator inside a tube of a magnetic resonance tomograph is provided. In this way, an extremely compact unit is possible.

In principle, the arrangement of the syringe near the magnetic resonance tomograph can be as shown, for example, in FIG. 9 of DE 10 2006 006 952 A1, in which a housing of the magnetic resonance tomograph is provided with an affixed securing part.

### BRIEF DESCRIPTION OF THE DRAWINGS

Further advantages and features of preferred embodiments of the invention are explained below with reference to the attached drawings, in which:

FIG. 1 shows a schematic perspective view of a typical embodiment of an injection device;

FIG. 2 shows a schematic perspective view of a further typical embodiment of an injection device; and

FIG. 3 shows a schematic perspective view of a typical embodiment of a magnetic resonance tomograph.

### DESCRIPTION OF PREFERRED ILLUSTRATIVE EMBODIMENTS

Typical embodiments are described below with reference to the figures. The invention is not limited to the illustrative embodiments; rather, the scope of the invention is defined by the claims.

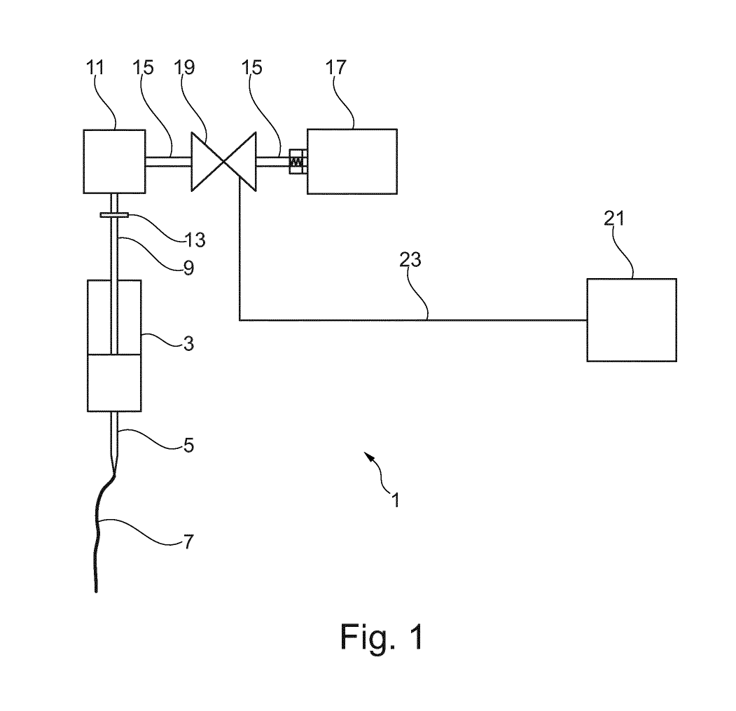

FIG. 1 shows a schematic perspective view of a typical embodiment of an injection device 1.

The injection device comprises a syringe with a reservoir 3 for holding a liquid. A nozzle 5 is arranged on the reservoir, and an injection hose 7 is secured on said nozzle 5. Liquid from the reservoir 3 can be injected through the nozzle 5 and the injection hose 7 into a patient. For this purpose, a plunger 9 of the syringe is moved in the reservoir 3, such that the space for the liquid becomes smaller.

In the illustrative embodiment in FIG. 1, the plunger 9 is driven by a drive member 11, which is pneumatic. The pneumatic drive member 11 comprises a spindle or a bolt 13 for driving the plunger 9, wherein the bolt 13 can be forced out of the housing of the drive member 11 by means of air pressure. The drive member 11 is connected via a fluid line 15 to a non-electrical energy accumulator 17 designed as a store for compressed gas.

The fluid line 15 is interrupted by a valve 19, with which the fluid line 15 can be shut off. The valve 19 can be controlled electromagnetically by a control device 21 via a cable 23. Although a certain amount of magnetically conductive material is brought into the environment of a magnetic resonance tomograph by the electromagnetically operated valve 19, this amount of material is low enough that the forces for a fastening mechanism can be overcome.

Advantages of the invention lie in its simple and reliable structure and in the possibility of remote control. In further illustrative embodiments, remote control takes place by air pressure, wherein the pressures for controlling the valve can be less than the pressure that prevails in the store for compressed gas.

In the illustrative embodiment in FIG. 1, the energy accumulator 17 is connected to the fluid line 15 via a thread 25. This permits a small energy accumulator and rapid exchangeability.

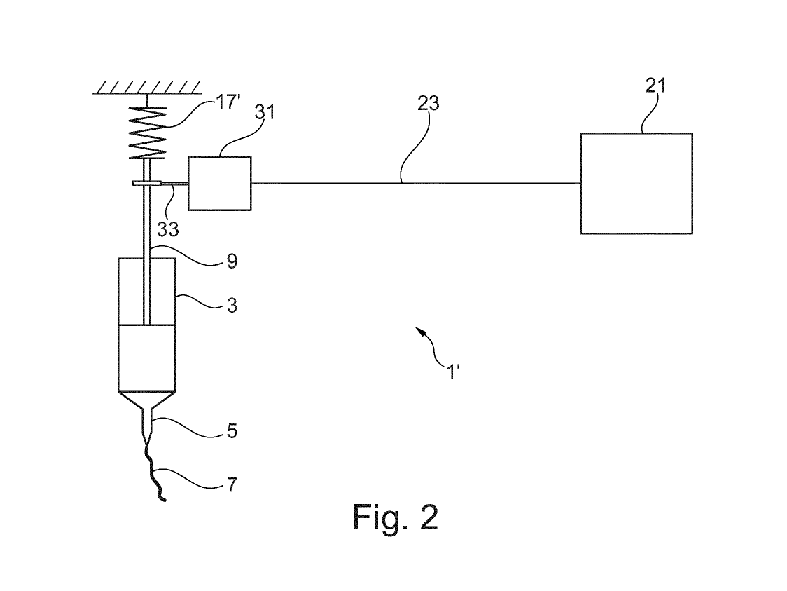

FIG. 2 shows a further typical illustrative embodiment of an injection device 1′, which has parts that are partially identical or similar to those in the injection device 1 of FIG. 1. Such parts are designated by the same reference signs and possibly not described again.

The injection device 1′ in FIG. 2 comprises a syringe with a reservoir 3, a nozzle 5 and a plunger 9. In the illustrative embodiment of the injection device 1′ in FIG. 2, the plunger 9 is subjected to pressure by a spring serving as energy accumulator 17′, wherein pressure can be applied only after release by an actuator 31. The actuator 31 comprises a pin 33 which can be driven in and out, for example via a small electromagnetic activation coil.

In typical illustrative embodiments, the actuator comprises electromagnetic material, but the amount is so low that the forces caused by it are manageable. The spring of the energy accumulator can also be made of metal, preferably a non-magnetizable metal. In this way, forces in the injection device are reduced during operation of the magnetic resonance tomograph. In further illustrative embodiments, the spring of the energy accumulator is made of plastic.

The actuator 31 is in turn connected to a control device 21 via a control line 23.

In typical illustrative embodiments, the control device is arranged outside a room in which the magnetic resonance tomograph is arranged. This permits remote control from outside the magnetic resonance tomography room.

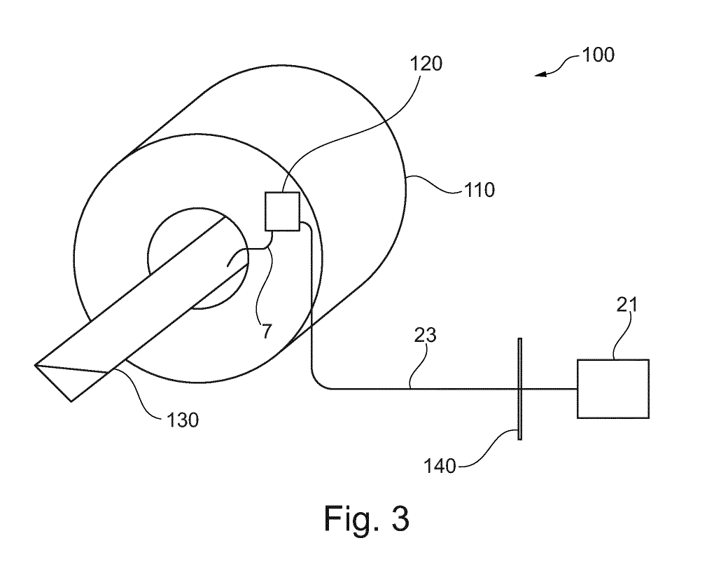

FIG. 3 shows an illustrative embodiment of a typical resonance tomograph 100, which is provided with an injection device in one of the typical embodiments described here. The magnetic resonance tomograph 100 has one or more coils received in a housing 110. A securing part 120, in which parts of an injection device as are described in FIG. 1 or 2 are arranged, is arranged on the housing 110 of the coils. For example, the syringe, on which the injection hose 7 is secured, is arranged in the securing part 120.

The injection hose 7 can be used to attach a patient, placed on a table 130, to the syringe and thus to the reservoir (reference sign 3 in FIGS. 1 and 2). An actuator (reference sign 31 in FIG. 2) arranged in the securing part 120, or a valve (reference sign 19 in FIG. 1) arranged in the securing part 120, can be actuated by way of the control line 23. A control device 21 connected via the control line 23 can be arranged outside a boundary wall 140 of a room in which the housing 110 of the coils is arranged along with the bench 130 located therein. Such a room is typically also referred to as an MRT room. The control device permits remote control from outside the MRT room.

In some embodiments, a seat for the syringe and a seat for the energy accumulator are provided in a housing of the coils of the magnetic resonance tomograph. This permits a compact structure and can make an additional housing for the injection device superfluous.

The invention has been described on the basis of illustrative embodiments and with the help of drawings. However, the invention is not limited to the illustrative embodiments. Rather, the scope of the invention is defined by the claims. Numerous modifications of the illustrative embodiments are possible within said scope, in particular with the features described herein.

1. An injection device, for injecting a liquid into a body in tomography, comprising:
a syringe with a reservoir for holding a liquid, with a nozzle arranged on the reservoir, and with a plunger for expelling the liquid from the reservoir through the nozzle,
a drive member, which is connected to the plunger in order to drive the plunger, and
a non-electrical energy accumulator, which is connected to the drive member in order to operate the drive member.

2. The injection device as claimed in claim 1, wherein the energy accumulator comprises a store for compressed gas.

3. The injection device as claimed in claim 2, wherein the energy accumulator is connected to the drive member via a fluid line.

4. The injection device as claimed in claim 1, with a valve, which is arranged between the energy accumulator and the drive member.

5. The injection device as claimed in claim 4, wherein the valve can be controlled electromagnetically.

6. The injection device as claimed in claim 1, wherein the distance from the energy accumulator to the drive member is less than 1 m.

7. The injection device as claimed in claim 1, wherein the energy accumulator comprises a spring, which is connected to the plunger.

8. The injection device as claimed in claim 7, wherein the spring can be released by means of an electromagnetic actuator.

9. A tomograph, in particular a computed tomograph or magnetic resonance tomograph, with the injection device as claimed in claim 1.

10. The tomograph as claimed in claim 9, which is designed as a magnetic resonance tomograph, wherein the syringe and the energy accumulator are arranged on a housing of coils of the magnetic resonance tomograph.

11. The injection device as claimed in claim 3, wherein the drive member effects a movement of the plunger with compressed gas from the fluid line.

12. The injection device as claimed in claim 1, wherein the tomography comprises magnetic resonance tomography or computed tomography.
